(19) United States
(12) Patent Application Publication
(10) Pub. No.: US 2016/0317751 A1
(43) Pub. Date: Nov. 3, 2016

(54) Mechanical Dose Expelled Indicator

(71) Applicant: NOVO NORDISK A/S, Bagsvaerd (DK)

(72) Inventor: Mads Groenlund Andersen, Copenhagen NV (DK)

(21) Appl. No.: 15/105,204

(22) PCT Filed: Dec. 18, 2014
(86) PCT No.: PCT/EP2014/078495

§ 371 Date: Jun. 16, 2016

(30) Foreign Application Priority Data
Dec. 20, 2013 (EP) 13199030.1

(51) Int. Cl. A61M 5/315; A61M 5/24; A61M 5/20; A61M 5/28

(52) U.S. Cl. CPC A61M 5/31553; A61M 5/31585; A61M 5/282; A61M 5/2425; A61M 5/31566; A61M 5/20; A61M 2005/3126

(57) ABSTRACT

The present invention concerns a dosing unit for an injection device (100) and an injection device incorporating the dosing unit. The dosing unit comprises a housing (102) extending along a longitudinal axis from a proximal housing end to a distal housing end, the distal housing end being adapted for connection with a variable volume reservoir, an injection mechanism for expelling a dose of a substance held in a connected variable volume reservoir, the injection mechanism being arranged at least partially in the housing (102) and comprising an injection button (157), a dose defining structure (140) configured to move from a start position to an end-of-dose position in response to a dose expelling operation of the injection button (157), and a piston rod (160) configured to move in a dose delivery direction from a first position to a second position in response to a movement of the dose defining structure (140) from the start position to the end-of-dose position, and a dose delivered indicator (109) configured to move axially in a distal direction from a dose ready position to a dose delivered position in response to the movement of the dose defining structure (140) from the start position to the end-of-dose position, wherein during movement from the dose ready position to the dose de livered position a portion of the dose delivered indicator (109) emerges increasingly from the distal housing end.

### FIELD OF THE INVENTION

The present invention relates to dose delivery devices for administration of medicinal substances.

### BACKGROUND OF THE INVENTION

Most modern devices for multiple dose delivery of pharmaceutical substances comprise a dose setting mechanism that allows a user to selectively set a dose to be dispensed from a substance containing reservoir. Some treatment regimens require setting and administration of a dose of drug that varies over time, whereas other treatment regimens require repeated setting and administration of a fixed dose of drug. In some cases the fixed dose may need to be adjusted over time, for example during a dosage titration period.

A common type of drug delivery device is the so-called pen injector which is a pen-shaped injection device used for intermittent subcutaneous administration of an active agent. Typically, such a pen injector allows the user to turn a dial about a general longitudinal axis to set a desired dose. If by accident too high a dose is set the dial may be reversed and the dose dialled down until the correct dose is reached. A pen injector may be a purely mechanical construction or it may be an electro-mechanical device, e.g. comprising an electronic memory module.

To be in compliance, as well as to avoid overdosing, it is important to pay particular attention during the dose setting procedure. In everyday life a busy schedule may give rise to negligence towards the various preparations leading up to a drug administration, in particular for people who are seasoned in self-administration. If the device used for administration of the drug does not offer an automatic storing of the ejected dose an inattentive user may be left in doubt after the administration as to the size of the dose actually received.

Accordingly, for drug delivery devices of the type which do not have an electronic memory it is desirable to provide a solution that enables the user to see, subsequent to a dose administration, which dose was just delivered from the device.

U.S. Pat. No. 3,905,366 (Callahan et al.) discloses a syringe for injection of medicinal liquids which syringe has means for determining the quantity delivered during injection. The syringe has a scale and a comparison mark arranged for relative displacement so that they may be reset to zero before each injection so as to permit a determination of each dose. For example, a scale carrier is slidably arranged on a vial adapted for insertion into a syringe frame such that the zero value may be brought in line with the front edge of a piston before an injection is commenced.

The position of the front edge of the piston relative to the scale after the injection thus directly informs the user of the dose expelled. However, the reliability of this dose expelled indication stands or falls with the precision with which the user positions the scale relative to the piston in the first place. An inattentive user may position the scale such that the zero value is somewhat offset from the front edge of the piston, inevitably resulting in a misleading post dose reading.

US 2012/0046613 (Sanofi-Aventis Deutschland GmbH) discloses a more modern type of drug delivery device in the form of a cartridge based pen injector. A piston in the cartridge is movable by a piston rod to expel doses of a contained drug. The piston rod is provided with consecutive numbers along its axis to indicate, through a window aperture in the device housing, delivered dose or remaining dose left in the cartridge.

While this device allegedly provides for an indication of a delivered dose because a dose related number on the piston rod is visible through the window aperture when the piston rod is halted, it appears that after the very first delivered dose indication every subsequent indication will reflect an accumulated dose rather than a delivered dose, as the piston rod successively advances in the cartridge, driving the piston towards the distal end thereof. This means that for each subsequent dose administration the user is required to remember the previously displayed number and to subtract this previously displayed number from the currently displayed number to obtain the indication of the dose delivered. Furthermore, since the dose related numbers are arranged on the piston rod and the piston rod only moves in one direction for the emptying of the cartridge it appears impossible with this construction to reset the numbers providing the dose related indication such that every post dose indication directly reflects the dose delivered.

### SUMMARY OF THE INVENTION

It is an object of the invention to eliminate or reduce at least one drawback of the prior art, or to provide a useful alternative to prior art solutions.

In particular, it is an object of the invention to enable the provision of a drug delivery device offering a dose expelled indication which incontestably reflects an actually delivered dose, regardless of the level of attention of the user during setting and/or administration of the dose.

It is a further object of the invention to provide such a device which consistently conveys a direct indication of the dose delivered, i.e. which enables a post dose reading of the just delivered dose after each dose administration.

In the disclosure of the present invention, aspects and embodiments will be described which will address one or more of the above objects and/or which will address objects apparent from the following text.

In a drug delivery device embodying the principles of the invention and comprising a housing extending along a longitudinal axis, and a dose delivery mechanism for delivering drug from a variable volume reservoir, the dose delivery mechanism comprises an actuator movable in a dose delivery direction from a first position to a second position in order to effect delivery of a dose of the drug, and a dose delivered indicator operatively coupled with the actuator during dose delivery and configured to move axially in a distal direction from a dose ready position to a dose delivered position in correlation with the actuator's movement from the first position to the second position, and at least during a final part of the distal movement of the dose delivered indicator a portion thereof extends from the housing. At least one dose related indicium may be arranged immovably with respect to the housing, whereby the dose delivered indicator visibly changes position relative to the at least one dose related indicium during movement from the dose ready position to the dose delivered position.

The drug delivery device may e.g. be an injection device for delivering a bolus dose of drug, an infusion device for delivering a dose of drug over a prolonged period of time, an inhaletion device for delivering a dose of drug to the respiratory system, or indeed any type of drug administration device capable of expelling a substance from a variable volume reservoir.

In one aspect of the invention a dosing unit for an injection device as defined by claim 1 is provided.

A dosing unit is thus provided comprising: a housing extending along a longitudinal axis from a proximal housing end to a distal housing end, where the distal housing end is adapted for being connected with a variable volume reservoir (such as e.g. a cartridge with a slidable piston and a self-sealing penetrable septum, or a pouch), and an injection mechanism for expelling a dose of substance from the variable volume reservoir, when the variable volume reservoir is connected with the distal housing end. The injection mechanism comprises an injection button, e.g. protruding from the proximal housing end, a dose defining structure which is adapted to move from a start position to an end-of-dose position in response to a specific operation of the injection button, and a piston rod adapted to move in a dose expelling direction from a first position to a second position in response to a movement of the dose defining structure from the start position to the end-of-dose position. The specific operation of the injection button (which may e.g. include a depression of the injection button against the housing) can thus be termed a dose expelling operation, as the movement of the piston rod from the first position to the second position is configured to reduce the volume of a variable volume reservoir connected with the housing and accordingly cause an expelling of substance therefrom.

An injection mechanism as the above described is commonly known in various forms in the art of injection devices. For example, in WO 2009/092807 a dose defining structure is disclosed which comprises a piston rod drive element that, responsive to a depression of an injection button and a resulting release of a spring, moves along the longitudinal axis of the injection device from one plateau to another, slaving the piston rod. The distance between the two plateaus thus defines the size of the dose being expelled. In WO 2006/045528 a rotatable piston drive member causes a specific helical advancement of a piston rod which is determined by the respective start and end positions of a helically displaceable dose indicator barrel being coupled thereto. The drive member rotates automatically when a torsion spring is released by manipulation of a dedicated locking member. Further examples are disclosed in e.g. WO 99/38554, WO 2005/039676 and WO 2012/089616.

The dosing unit further comprises a dose delivered indicator which is configured to move axially in a distal direction (i.e. away from the proximal housing end) from a dose ready position, in which a dose of drug is ready to be expelled, to a dose delivered position, in which the dose of drug has been expelled, in response to the movement of the dose defining structure from the start position to the end-of-dose position. During the movement from the dose ready position to the dose delivered position a portion of the dose delivered indicator emerges increasingly from the distal housing end.

The emergence of the dose delivered indicator from the distal housing end is a verification that the piston rod has actually undergone a movement in the dose expelling direction relative to the housing, and the degree of visibility of the dose delivered indicator is directly correlated with the distance travelled by the piston rod, enabling an estimation or a determination of the dose expelled from a connected variable volume reservoir.

The dose delivered indicator may form part of the injection mechanism and be operatively coupled with the piston rod during dose expelling such that a driving force is imposed on the piston rod in response to the dose delivered indicator undergoing relative motion with respect to the housing. For example, rotational motion may be imparted to the piston rod, e.g. via an intermediate component, when the dose delivered indicator undergoes translational motion relative to the housing. In that case the piston rod may be threadedly engaged with a nut member arranged fixedly in the housing, whereby the rotational motion is converted to a helical advancing motion relative to the housing.

If the dose delivered indicator forms part of the injection mechanism a minimum number of components is needed to obtain the post dose indication.

The dosing unit may be designed for manual or automatic actuation of the piston rod. In case of the latter, an energy providing unit, such as e.g. a spring structure, may be arranged to bias the dose delivered indicator in the distal direction. Such an energy providing unit may then be activatable to convey stored energy to the piston rod and thereby cause a dose expelling which is practically effortless to the user.

The dosing unit may be adapted to form part of an injection device of the prefilled and disposable type, i.e. where a variable volume reservoir, such as a cartridge, is permanently coupled with the housing, or of the loadable, e.g. re-useable type, i.e. where a variable volume reservoir, such as a cartridge, can be coupled with the housing by a user in a repeatable manner, e.g. via a displaceable reservoir holder adapted to receive and releasably retain the variable volume reservoir. The injection device may even be a single shot injector which is configured to deliver only one, predetermined (e.g. by the manufacturer) or settable, dose of drug from a pre-coupled or loaded variable volume reservoir.

At least one dose related indicium, such as e.g. a number or a line or a colour change, may be provided on the portion of the dose delivered indicator which emerges increasingly from the distal housing end during the movement of the dose delivered indicator from the dose ready position to the dose delivered position. This will enable the user to verify that a certain dose has been expelled. For example, if the injection device is a fixed dose device capable of expelling one or more doses of a predetermined size a single line or a similar form of indication may be arranged on the dose delivered indicator at a position which becomes visible exteriorly of the housing only when the predetermined dose has been expelled.

In particular embodiments of the invention, e.g. related to dosing units for injection devices which offer setting and delivery of a plurality of different doses, the at least one dose related indicium comprises a scale which indicates progression in the proximal direction. The scale is provided on the portion of the dose delivered indicator which is able to emerge increasingly from the distal housing end and is arranged in correlation with the resulting distance travelled by the piston rod. Hence, as the dose delivered indicator emerges increasingly from the distal housing end during dose expelling the scale displays indicia which indicate a gradual increase of the dose delivered. At the end of the dose delivery the scale may e.g. be used to directly read off a number which then corresponds to the size of the dose that was expelled.

The dose delivered indicator may further be movable from the dose delivered position to the dose ready position. During this reverse movement the dose delivered indicator is decoupled from the piston rod, whereby the actuator remains in position while the dose delivered indicator is being reset. This feature is especially useful if the dosing unit is for use in a multishot injection device which is capable of expelling a plurality of doses from a pre-coupled or loaded variable volume reservoir, because it provides for a direct indication of the last delivered dose after each dose administration, which is far more convenient to the user than indications of accumulated doses, as the user needs neither remember the last indicated number during the dose administration nor subtract it subsequently from the new indicated number.

The dosing unit may further comprise a dose setting mechanism for defining the extent of movement of the piston rod from the first position to the second position. The dose setting mechanism may comprise the dose defining structure and a dose setting button operable to move the dose defining structure relative to the housing, e.g. to bring the dose defining structure to the start position. The dose defining structure may comprise dose setting indicia for indicating the size of a set dose, and the housing may comprise a window for inspection of at least one of the dose setting indicia at a time. In particular embodiments of the invention the dose defining structure comprises a scale drum having the dose setting indicia arranged helically on an exterior surface portion.

At least a subset of the dose setting indicia may successively pass by the window during dose delivery, thereby continuously indicating a remaining dose to be delivered. This allows the user to verify that the dose delivery is progressing properly.

In another aspect of the invention an injection device is provided comprising a dosing unit as described above, a variable volume reservoir comprising an outlet and a movable wall, such as e.g. a cartridge sealed by a penetrable self-sealing septum and a slidable piston, and a reservoir holder for retaining the variable volume reservoir in an axially fixed position relative to the distal housing end, in which position the piston rod abuts the movable wall.

The increasing emergence of the dose delivered indicator from the distal housing end allows the user to visually inspect the progression of the dose expelling from the variable volume reservoir. In the end-of-dose position the dose has been expelled completely (or as completely as physically possible), and the position, or the extent, of the visible portion of the dose delivered indicator serves to indicate that the dose expelling has been successfully carried out (in case the variable volume reservoir is an elastomeric piston the entire dose may not be completely expelled until a few seconds after the dose delivered indicator has reached the end-of-dose position and the piston, which is being compressed during its advancement, has relaxed). Hence, notably, the degree of visibility of the dose delivered indicator correlates with the amount of drug having been expelled from the variable volume reservoir. This also enables the user to check if the intended dose has actually been delivered, or if e.g. a clogging of the fluid pathway from the variable volume reservoir to the injection site has occurred.

The reservoir holder may be a separate element which is configured for axial fixation with respect to the distal housing end, and which is capable of receiving and axially fixing the variable volume reservoir with respect to the housing, e.g. like a cartridge holder known in the art of pen injection devices for the treatment of diabetes. Such a cartridge holder is typically provided with a cartridge receiving space and a needle mounting portion for receiving a needle assembly in such a manner that a portion of an injection needle penetrates the self-sealing septum of the cartridge when the needle assembly is mounted on the needle mounting portion. Alternatively, the reservoir holder may be integrated in the housing.

The injection device may further comprise at least one dose delivered indicium arranged on the variable volume reservoir, or on the reservoir holder. The at least one dose delivered indicium is immovably positioned distally of the distal housing end, when the variable volume reservoir is connected with the housing, so as to allow the user to inspect the relative position of the dose delivered indicator and the at least one dose delivered indicium following a dose delivery procedure.

The at least one dose delivered indicium may comprise a line, a numeral, a letter, a colour change, or any other visible and/or tactile indication. The at least one dose delivered indicium may be arranged to be approached, and potentially reached, by a portion of the dose delivered indicator, such as e.g. the end portion of the dose delivered indicator, during the distal movement of the dose delivered indicator from the dose ready position to the dose delivered position. In case a scale is provided on the dose delivered indicator the at least one dose delivered indicium may be arranged to provide for a reading of the scale.

Particularly, the at least one dose delivered indicium may comprise a dose delivered scale arranged on the variable volume reservoir or on the reservoir holder, the dose delivered scale indicating progression in the distal direction, and the dose delivered indicator may be configured to slide along the dose delivered scale during its movement from the dose ready position to the dose delivered position. Thereby, the dose delivered indicator serves as a bar which indicates a level on the dose delivered scale that corresponds to the dose having been expelled from the variable volume reservoir. Thereby, a mechanical dose expelled indicator is provided which is intuitively read as it resembles the well-known mercury-in-glass thermometer.

The dose delivered scale may comprise numerals indicating a range which corresponds to, e.g. equals, the range indicated by the dose setting indicia on the dose defining structure. This will provide a complete correspondence between the scale used for the dose setting and the scale used for the reading of the delivered dose.

In some embodiments of the invention the reservoir holder comprises a body which extends distally from the distal housing end along the longitudinal axis, and a longitudinal groove in the body which groove is configured for sliding reception of the dose delivered indicator. Such a solution provides for a slender injection device construction which may be attractive to some users.

The injection device may further comprise a protective cap mountable onto, and dismountable from, the housing. The protective cap may be structured to interface with the dose delivered indicator and move the dose delivered indicator from the dose delivered position to the dose ready position during mounting of the protective cap onto the housing after dose delivery. Thereby, since the mounting of the protective cap following a dose administration is part of the customary handling procedure for this type of drug delivery device an automatic resetting of the dose delivered indicator is obtained without an introduction of any additional handling steps.

For example, the protective cap may be adapted to be mounted onto the distal housing end to cover the variable volume reservoir and/or the reservoir holder. The mounting of the protective cap onto the distal housing end may involve at least a relative axial motion between the protective cap and the housing, such as e.g. a relative translation or a relative helical motion. The protective cap may comprise a first interface portion and the dose delivered indicator may comprise a second interface portion adapted for interaction with the first interface portion during a portion of the at least a relative axial motion between the protective cap and the housing. In particular, the first interface portion may comprise a rim, and the second interface portion may comprise a distal abutment surface, whereby the rim will abut the distal abutment surface and urge the dose delivered indicator proximally during mounting of the protective cap onto the distal housing end.

In the present context the term “piston rod” denotes an actuator device of the type commonly known in the art of injection devices, i.e. an elongated structure capable of exerting a driving force on a movable wall of a reservoir. It is emphasized that the “piston rod” may include a distal coupling element, sometimes referred to in the art as a “piston rod foot” or a “piston washer”.

Further, in the present context, any statement related to the cap being mounted on or onto the housing should be interpreted to cover both the cap being mounted on or onto the housing and the cap being mounted on or onto a part coupled with the housing, such as e.g. a reservoir holder. When the cap is mounted onto the distal housing end it means that the cap is mounted onto the housing at the distal housing end.

In the present specification, reference to a certain aspect or a certain embodiment (e.g. “an aspect”, “a first aspect”, “one embodiment”, “an exemplary embodiment”, or the like) signifies that a particular feature, structure, or characteristic described in connection with the respective aspect or embodiment is included in, or inherent of, at least that one aspect or embodiment of the invention, but not necessarily in/of all aspects or embodiments of the invention. It is emphasized, however, that any combination of the various features, structures and/or characteristics described in relation to the invention is encompassed by the invention unless expressly stated herein or clearly contradicted by context.

The use of any and all examples, or exemplary language (e.g., such as, etc.), in the text is intended to merely illuminate the invention and does not pose a limitation on the scope of the same, unless otherwise claimed. Further, no language or wording in the specification should be construed as indicating any non-claimed element as essential to the practice of the invention.

### BRIEF DESCRIPTION OF THE DRAWINGS

In the following the invention will be further described with references to the drawings, wherein

FIG. 1 is a perspective view of a portion of an injection device according to an embodiment of the invention,

FIG. 2 is a perspective view detailing elements of a drive mechanism in the injection device,

FIGS. 3-7 are perspective views of the portion of the injection device in different states during use, and

FIGS. 8a-c are perspective views of the entire injection device in different states during an injection.

In the figures like structures are mainly identified by like reference numerals.

### DESCRIPTION OF EXEMPLARY EMBODIMENTS

When in the following relative expressions, such as e.g. “upwards” and “downwards” and “clockwise” and “counter-clockwise”, are used these refer to the appended figures and not necessarily to an actual situation of use. The shown figures are schematic representations for which reason the configuration of the different structures as well as their relative dimensions are intended to serve illustrative purposes only.

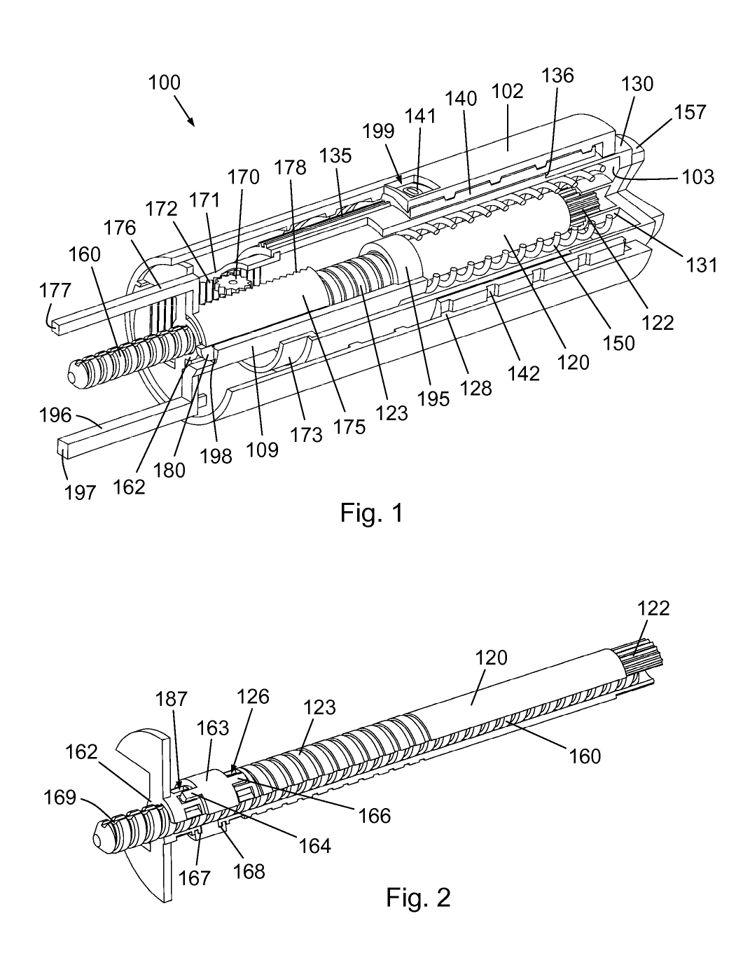

FIG. 1 is a perspective view of a portion of an injection device 100 according to an embodiment of the invention, specifically of a proximal portion of the injection device 100, carrying a dose engine. The injection device 100 is in a pre-use state and portions of some elements thereof have been removed from the figure to provide a detailed overview of the construction.

The injection device 100 is of the so-called pen injector type and has a tubular housing 102 extending along a longitudinal general axis and accommodating a number of functional components. The housing 102 is coupled with a drug containing cartridge (not shown) in a manner conventionally known in the art, i.a. meaning that the cartridge during use of the injection device 100 is at least axially fixed with respect to the housing 102. Central to the function of the injection device 100 is an axially extending piston rod 160 which is in threaded engagement with a nut 162 that is both axially and rotationally fixed in the housing 102.

The distal end portion of the piston rod 160 is coupled to a piston (not shown) in the cartridge such that any advancing axial motion of the piston rod 160 is transferred to the piston, essentially for pressurisation of the cartridge, as is also conventionally known in the art.

It is noted that all rotational movements described in the below and referred to as clockwise or counter-clockwise are described as seen from the distal end of the piston rod 160 (i.e. from left to right in FIG. 1).

The housing 102 is provided with an interior thread 128 which cooperates with an exterior helical track segment 142 on a scale drum 140, allowing the scale drum 140 to undergo a well-defined helical motion in the housing 102. The scale drum 140 carries a plurality of dose indicia 141 for indicating to a user the particular size of a set dose. The dose indicia 141 are successively viewable through a window 199 in the housing 102 when the scale drum 140 travels along the interior thread 128 e.g. from a proximal “zero dose” position to a distal “maximum dose set” position. The proximal “zero dose” position is defined by a proximal stop surface (not visible) providing a rotational stop for proximal motion of the scale drum 140 at the proximal end of the interior thread 128, whereas the “maximum dose set” position is defined by a distal stop surface (not visible) providing a rotational stop for distal motion of the scale drum 140 at the distal end of the interior thread 128.

The scale drum 140 is rotationally locked to a dial 130 via a longitudinal interior projection 144 (see FIG. 4) and an axially extending spline 135 on the exterior surface of the dial 130. While rotationally interlocking the scale drum 140 and the dial 130 this splined connection allows relative axial motion between the two. The dial 130 is at its distal end portion axially locked to a coupling piece 173 which comprises an axially aligned leg 171 with a radially inwardly facing toothed surface 172. At the proximal end portion of the dial 130 a push button 157 is arranged, which is axially locked to but rotationally decoupled from the dial 130, and the two together serve as an injection button. Further, a sleeve 131 extends axially from an inner end face 103 of the dial 130. The sleeve 131 has a toothed inner surface and is configured to be brought into and out of rotational interlocking engagement with a toothed end portion 122 of a dose preparation tube 120 which extends axially within the housing 102.

The dose preparation tube 120 has a threaded end portion 123 opposite the toothed end portion 122. The threaded end portion 123 interfaces with a drive nut 195 in a non-self-locking thread engagement. The drive nut 195 forms part of an actuation rod 109, the function of which will be described in detail below. The actuation rod 109, which is axially displaceable but rotationally fixed with respect to the housing 102, has a longitudinal extension 196 which ends in an abutment face 197. The longitudinal extension 196 is transversally offset from a main portion of the actuation rod 109 and is adapted to slide along a cartridge holder (not shown in FIG. 1) both during dose delivery and dose preparation. The cartridge holder is attached to a distal portion of the housing 102 and serves to hold and protect the cartridge in a manner conventionally known in the art.

A pre-tensioned compression spring 150 is arranged to act between the inner end face 103 and the actuation rod 109, constantly biasing the dial 130 and the push button 157 proximally, out of the housing 102, and the actuation rod 109 distally. In the shown pre-use state of the injection device 100 distal motion of the actuation rod 109 is prevented by a lock member 180 abutting a transversal surface 198 of the actuation rod 109. The lock member 180 is pivotally arranged on the nut 162 but is in FIG. 1 prevented from pivoting by an edge portion of a button coupling rod 175 which is axially displaceable but rotationally fixed with respect to the housing 102. The button coupling rod 175 has a toothed straight edge 178, which is in engagement with a transmission wheel 170, and a longitudinal extension 176, which is transversally offset from the toothed straight edge 178 and which ends in an abutment face 177. The transmission wheel 170 is further in engagement with the toothed surface 172, such that the coupling piece 173, the button coupling rod 175, and the transmission wheel 170 together provide a double rack and pinion drive.

In the present situation, given that the actuation rod 109 is prevented from undergoing distal motion in the housing 102 due to the lock member 180, the bias of the spring 150 on the dial 130 causes the dial 130 to exert a pulling force on the coupling piece 173 which then via the double rack and pinion structure is converted to a distal movement of the button coupling rod 175, unless a counter-acting force is applied to the abutment face 177. Although not shown in FIG. 1, in the depicted pre-use state of the injection device 100 a removable protective cap is securely mounted onto a cap receiving portion at the distal end portion of the housing 102 such that a portion of the cap abuts the abutment face 177 and resists the bias conveyed to the longitudinal extension 176, thereby maintaining the button coupling rod 175 in position. The injection device 100 is thus in fact stably locked in a tensioned state. As will be explained in more detail below once the retaining force on the abutment face 177 is removed the relaxation of the spring 150 will cause the dial 130 and the push button 157 to translate proximally. A stop surface 136 on the dial 130 limits the proximal motion of the dial 130 and the push button 157 relative to the housing 102.

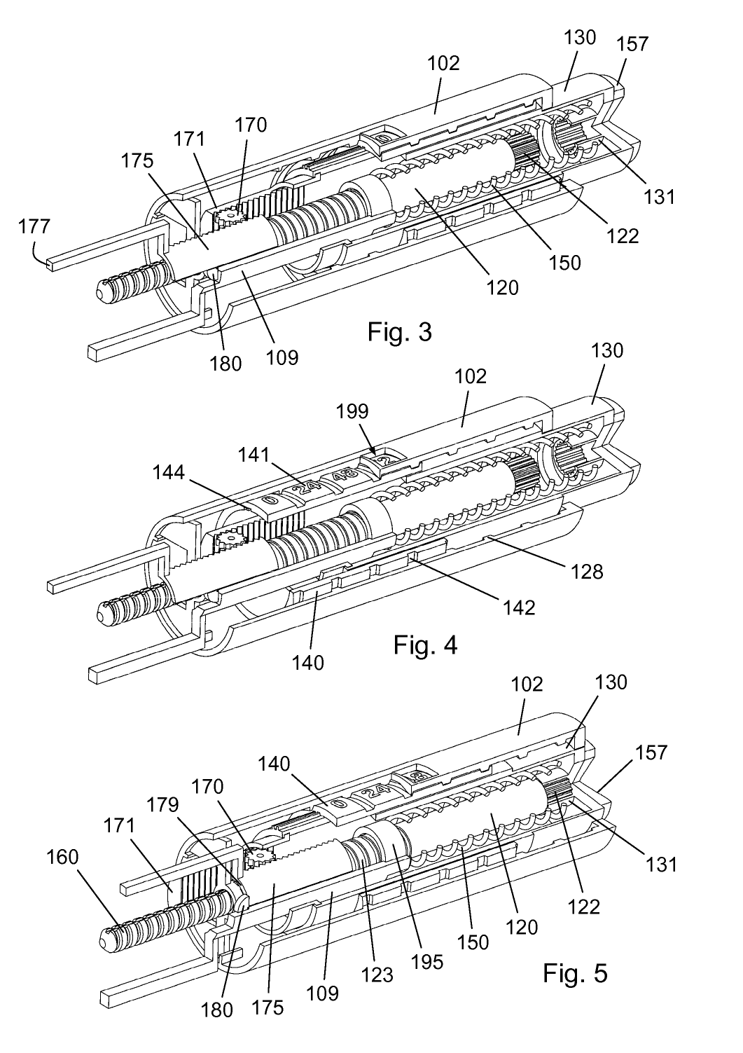

FIG. 2 is a detailed view of the piston rod advancement mechanism as employed in the injection device 100. A rotatable piston rod guide 163 couples the nut 162 and the dose preparation tube 120 via an inner groove 167 for axial interlocking connection with the nut 162 and an inner groove 168 for axial interlocking connection with the threaded end portion 123. The piston rod guide 163 has a distal pawl 164, which in combination with a plurality of circumferentially spaced apart indentations 187 on the nut 162 provide a distal ratchet mechanism, and a proximal pawl 166 which in combination with a plurality of circumferentially spaced apart indentations 126 on the dose preparation tube 120 provide a proximal ratchet mechanism. The distal ratchet mechanism allows clockwise rotation of the piston rod guide 163 relative to the nut 162 but prevents counter-clockwise rotation of the piston rod guide 163. The proximal ratchet mechanism allows relative rotation between the dose preparation tube 120 and the piston rod guide 163 when the dose preparation tube 120 is rotated counter-clockwise, but prevents relative rotation between the dose preparation tube 120 and the piston rod guide 163 when the dose preparation tube 120 is rotated clockwise. The double ratchet comprised of the distal ratchet mechanism and the proximal ratchet mechanism thus allows the dose preparation tube 120 to drag the piston rod guide 163 along in the clockwise direction and to rotate freely in the counter-clockwise direction while the piston rod guide 163 remains stationary.

The piston rod guide 163 further has a radially inwardly directed protrusion (not visible) for engagement with an axial groove 169 on the piston rod 160. The piston rod 160 and the piston rod guide 163 are thus rotationally interlocked but capable of relative axial motion.

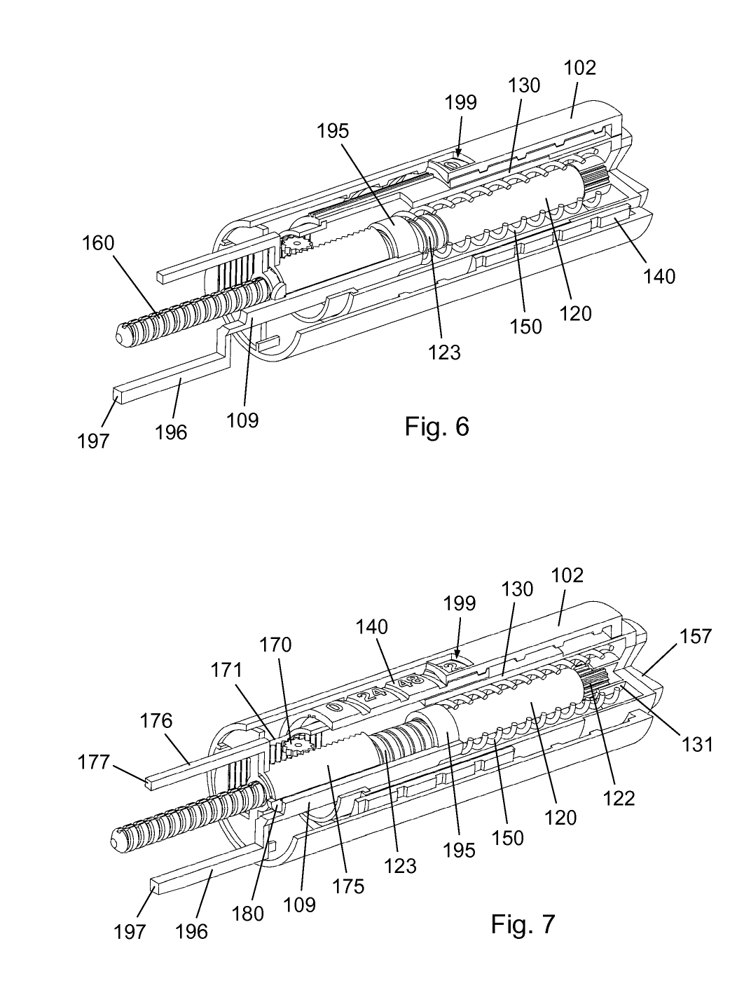

The functionality of the dose setting and delivery mechanisms will now be described with reference to FIGS. 3-7. When taking the injection device 100 into use the protective cap is firstly removed. This removes the retaining force on the abutment face 177 and allows the spring 150 to expand. The spring 150 thus urges the dial 130 with the push button 157 proximally until the stop surface 136 abuts the interior end wall of the housing 102, and the double rack and pinion drive accordingly forces the button coupling rod 175 a distance distally. The end result of this is illustrated by FIG. 3.

The proximal motion of the dial 130 also causes the sleeve 131 to disengage from the toothed end portion 122. The dial 130 is thus now capable of being rotated without affecting the dose preparation tube 120. A dose is set by rotation of the dial 130 relative to the housing 102. Due to the spline connection between the dial 130 and the scale drum 140 and the threaded interface between the scale drum 140 and the housing 102 when the dial 130 is dialled counter-clockwise the scale drum 140 displaces helically downwards in the housing 102 in response, and when the dial 130 is dialled clockwise the scale drum 140 displaces helically upwards in the housing 102. In FIG. 4 the dial 130 has been dialled to set a dose of “72” units.

Dose delivery is executed by depression of the push button 157, as illustrated in FIG. 5. The push button 157 may actually be depressed a certain distance without causing more than a reversed motion of the double rack and pinion drive and a compression of the spring 150. A discontinuation of the depression force in this instance will simply cause the spring 150 to return the push button 157 to its proximal most position. However, once the button coupling rod 175, during its proximal displacement, reaches a specific axial position in the housing 102 an end surface 179 passes the fulcrum of the lock member 180 and the lock member 180 will be free to pivot, whereby the pre-tensioned spring 150 will be released and as a result force the actuation rod 109 distally. As the lock member 180 pivots to allow passage of the actuation rod 109 the button coupling rod 175 becomes prevented from distal motion in the housing 102 due to the lock member 180 being prevented from returning to the original position by the actuation rod 109. At this point if the user releases the pressure on the push button 157 the dial 130 will consequently be prevented from proximal motion and will thus stay inside the housing 102.

The depression of the push button 157 also leads to a rotational re-engagement of the sleeve 131 and the toothed end portion 122. This happens before the flipping over of the lock member 180, such that when the spring 150 is released and the actuation rod 109 is suddenly propelled distally the dial 130 and the dose preparation tube 120 are rotationally interlocked. Due to the threaded engagement between the drive nut 195 and the threaded end portion 123 the distal movement of the actuation rod 109 causes the dose preparation tube 120 to spin clockwise.

The clockwise rotation of the dose preparation tube 120 causes a clockwise rotation of the piston rod guide 163, due to the above described double ratchet mechanism, and thereby also of the piston rod 160. The threaded engagement between the piston rod 160 and the nut 162 thus results in a helical advancement of the piston rod 160, whereby the piston (not shown) is advanced axially in the cartridge (not shown) to expel a volume of drug through an attached injection needle (not shown). The volume expelled is determined by the position of the scale drum 140 in the housing 102 at the time of release of the spring 150 because the clockwise rotation of the dose preparation tube 120 also causes a clockwise rotation of the dial 130 and thereby of the scale drum 140, and the rotation of the three continues until the scale drum 140 meets the proximal stop surface which defines the “zero dose” position. This end-of-dose state of the injection device 100 is illustrated in FIG. 6.

It is noted that as the injection progresses the actuation rod 109 is moved further distally and the axial end position of the abutment face 197 corresponding to the “zero dose” position of the scale drum 140 is uniquely correlated with the distance travelled by the scale drum 140 from its position at release of the spring 150 to the proximal stop surface.

In the end-of-dose state of the injection device 100 the push button 157 is prevented from proximal motion and therefore has to stay depressed in the housing 102. Hence, it is not possible to set a dose at this point. It is common practice when handling injection devices to re-mount the protective cap following an injection. In the course of re-mounting the protective cap onto the cap receiving portion of the injection device 100 a portion of the cap, such as e.g. a segment of the cap rim or a protrusion, abuts the abutment face 197 and pushes the actuation rod 109 proximally with respect to the housing 102.

The resulting proximal movement of the drive nut 195 causes the dose preparation tube 120 to spin counter-clockwise, relative to the housing 102 but also relative to the piston rod guide 163 due to the double ratchet mechanism, so the piston rod 160 is left unaffected. The counter-clockwise rotation of the dose preparation tube 120 causes a corresponding counter-clockwise rotation of the dial 130 which leads to a downward helical displacement of the scale drum 140.

The proximal movement of the drive nut 195 also causes a compression of the spring 150 which is progressive until the actuation rod 109 reaches the axial position where the transversal surface 198 passes the fulcrum of the lock member 180. At this position of the actuation rod 109 the lock member 180 is free to pivot and thus no longer functions as a block for distal motion of the button coupling rod 175. So, as the spring 150 seeks to relax and constantly biases the inner end face 103 in the proximal direction, the dial 130 is urged proximally, pulling the coupling piece 173, and the double rack and pinion drive consequently urges the button coupling rod 175 distally, causing the lock member 180 to flip over and abut the transversal surface 198. The spring 150 will displace the dial 130 proximally a small distance until the abutment face 177 abuts the protective cap and further distal motion of the button coupling rod 175 thereby is prevented. This corresponds to the state of the injection device 100 shown in FIG. 7. In this state the lock member 180 stably prevents distal motion of the actuation rod 109, as it is prevented from pivoting by the button coupling rod 175. As long as the protective cap remains mounted on the cap receiving portion a depression of the push button 157 only leads to an additional compression of the spring 150 which has no effect on the secured injection mechanism. At termination of the push force the spring 150 will return to the slightly less compressed state shown in FIG. 7.

Notably, when the protective cap is re-mounted on the cap receiving portion the actuation rod 109 is returned to the exact same axial position within the housing 102 that it initially assumed before the dose ejection was commenced. Due to the threaded interface between the drive nut 195 and the threaded end portion 123 this means that the dose preparation tube 120 is consequently returned rotationally to the exact same angular position relative to the housing 102 that it initially assumed before the dose ejection was commenced. The dose preparation tube 120 has thus during re-mounting of the protective cap undergone the exact opposite rotation to the one it underwent during the dose delivery, and since the dose preparation tube 120 and the dial 130 are rotationally interlocked so has the dial 130. Consequently, due to the splined connection between the dial 130 and the scale drum 140 and the threaded connection between the scale drum 140 and the housing 102, the scale drum 140 has been returned to the position it assumed immediately before the push button 157 was depressed and the spring 150 was released. In other words, by the re-mounting of the protective cap onto the cap receiving portion a setting of the last ejected dose has automatically been performed.

In fact, every time the protective cap is mounted onto the cap receiving portion the dose preparation tube 120 will be returned, in the above described manner, to the initial angular position, which can be defined as a dose prepared position within the housing 102, thereby bringing the injection device 100 in a “DOSE PREPARED” state.

When the user dismounts the protective cap before the next injection the dial 130 and the push button 157 will re-protrude from the housing 102 and the sleeve 131 will disengage from the toothed end portion 122, as described above in connection with FIG. 3. The user can now either choose to simply position the injection device 100 at the desired skin site and press the push button 157 to deliver the same dose as was last delivered, or adjust the dose size by turning the dial 130 in the appropriate direction before performing the injection procedure.

In case the user chooses to adjust the dose, and thereby set a new dose, the scale drum 140 will change position within the housing 102 and assume a new position corresponding to the desired dose viewed through the window 199. Because the dial 130 is decoupled from the toothed end portion 122 the repositioning of the scale drum 140 will not affect the dose preparation tube 120. Only when the push button 157 is subsequently depressed and the sleeve 131 reengages with the toothed end portion 122 the scale drum 140 and the dose preparation tube 120 become coupled to undergo correlated movements relative to the housing 102, provoked by the spring 150, as previously described. During these correlated movements the scale drum 140 will again reach the “zero dose” position and abruptly stop further expansion of the spring 150 and distal motion of the actuation rod 109. When this happens the axial end-of-dose position of the abutment face 197 relative to the housing 102 will be different from its previous end-of-dose position and, consequently, the dose preparation tube 120 will have undergone a different angular displacement than the one it underwent during the previous dose delivery. Nevertheless, when the cap is re-mounted on the cap receiving portion the actuation rod 109 will once again be returned to the same axial position as before, since that axial position is defined by the position of the cap portion abutting the abutment face 197 relative to the housing 102 when the cap is securely mounted. Due to the engagement between the drive nut 195 and the threaded end portion 123 the reversed motion of the actuation rod 109 will lead to a reversed motion of the dose preparation tube 120, which will again lead to a reversed motion of the scale drum 140. Thereby, the dose preparation tube 120 is returned to the exact same angular position relative to the housing 102 that it assumed before the dose ejection (the dose prepared position), and the scale drum 140 is returned to the position in which the new dose is viewed through the window 199.

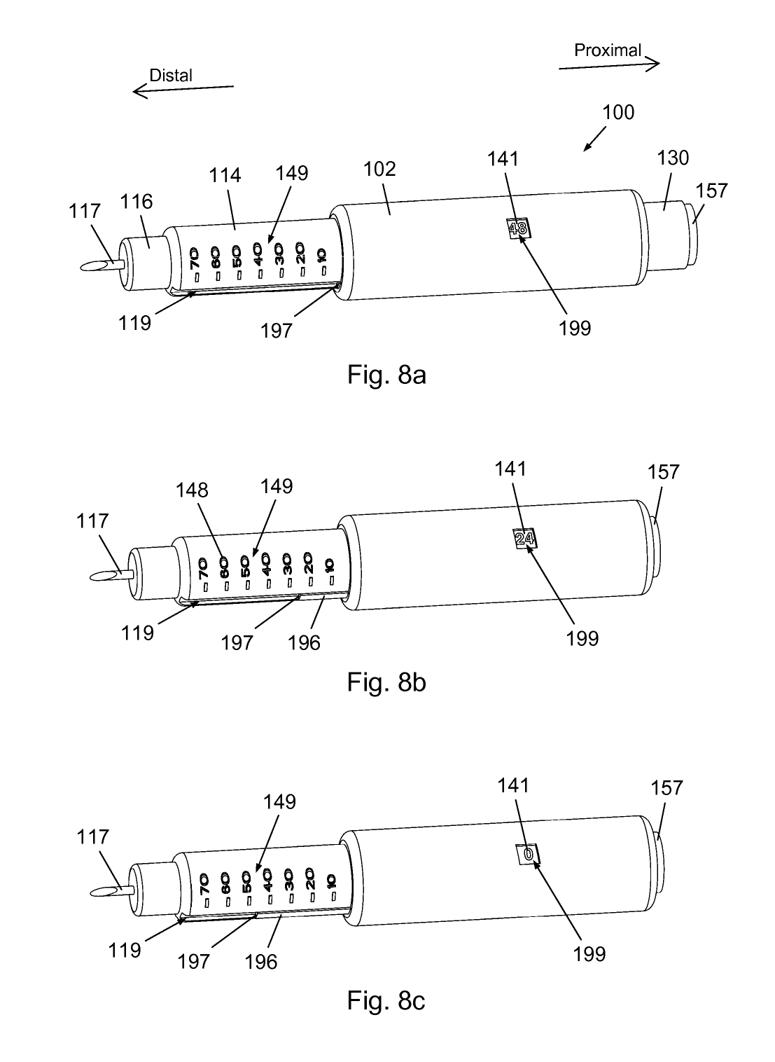

FIGS. 8a-8c show the entire injection device 100 in different states during dose setting and delivery with a view to further visualise the dose delivered indicator according to the present invention. A cartridge holder 114 is attached to the distal end of the housing 102. The cartridge holder 114 carries a cartridge (not visible) which holds a volume of a medical substance. A needle hub 116 is attached to the distal end of the cartridge holder 114. The needle hub 116 supports a double pointed injection needle 117 which in the current position provides for fluid communication with the interior of the cartridge, as conventionally known in the art. Numerals 148 are provided along the exterior surface of the cartridge holder 114, together forming a post dose scale 149. The post dose scale 149 is arranged next to an axial groove 119 that is angularly aligned with the longitudinal extension 196.

FIG. 8a depicts a situation just before depression of the push button 157. Following removal of the cap (not shown) the dial 130 has been turned and a dose of “48” units has been set, as seen through the window 199. The abutment face 197 is flush with the distal end of the housing 102 and therefore barely visible, indicating that no dose has been expelled.

FIG. 8b depicts a situation after depression of the push button 157, where the lock member 180 has flipped over to release the spring 150, and an ejection of the medical substance through the injection needle 117 is ongoing. The actuation rod 109 is displaced distally under the force of the spring 150, whereby the longitudinal extension 196 is slid distally in the axial groove 119. Through the window 199 the user can see how many units are left of the administration. At the same time the post dose scale indicates the number of units already expelled during the current dose delivery, because the axial position of the abutment face 197 is correlated with the position of the scale drum 140, as previously described.

In FIG. 8c the entire set dose has been delivered through the injection needle 117 and the scale drum 140 has reached the “zero dose” position. In this situation the post dose scale 149 reads “48” and thereby gives the user the opportunity to verify the size of the dose that was just delivered, a feature not previously available in all-mechanical injection devices. This opportunity provides an effortless additional control measure for the user to check her/his dose regimen compliance, a valuable extra tool to ensure that the dose received was within an acceptable range. When it comes to delivery of high potency drugs, such as e.g. insulin for the treatment of diabetes, it is crucial to avoid any uncertainty regarding the dosing.

When the protective cap is being re-mounted over the cartridge holder 114 and secured to the cap receiving portion a portion of the cap abuts the abutment face 197 and causes the longitudinal extension 196 to slide proximally in the axial groove back to the position indicated in FIG. 8a.

Apart from the dose delivered indicator and the automatic setting of the last ejected dose in response to a mounting of the protective cap the injection device 100 possesses a user interface which clearly indicates to its surroundings the possibilities of operation as well as a feed-forward to the next step in the use sequence. When the protective cap is on the injection device 100 the push button 157 is depressed which signals to the user that the system is “closed” or “passive” or “not to be operated”, simply because no operation of a dose setting button or an injection button is possible. When the protective cap is removed and the dial 130 emerges automatically from the housing 102, carrying the push button 157, this signals to the user that a following step is to either set or inject a dose of the medical substance (depending on whether an injection needle 117 is already in position on the cartridge holder 114). Both operators are ready for manipulation. When the push button 157 is eventually depressed to deliver a set dose the dial 130 is locked within the housing 102 due to the double rack and pinion drive and the arrangement of the lock member 180, signalling to the user that no further operation is required in connection with the current dose delivery procedure. When the protective cap is re-mounted the dial 130 and the push button 157 remains substantially depressed, and the injection device 100 is again “passive”.

Notably, in case the protective cap is off and the dial 130 protrudes from the housing 102 if the cap is re-mounted without the push button 157 having been depressed to release the spring 150 then the double rack and pinion drive will simply cause the dial 130 to move back into the housing 102 and thus re-establish the “closed” appearance of the injection device 100.

As is clear from the above the lock member 180 functions as a binary switch lock between the dose setting mechanism and the dose delivery mechanism in the sense that when the piston rod 160 is advanced during dose delivery no dose setting or adjustment is possible because the dial 130 is inoperably and inescapably contained within the housing 102, and when the dial 130 protrudes from the housing 102 and is manipulable by the user the spring 150 is securely retained by the immovable actuation rod 109, preventing any advancement of the piston rod 160.

The particular arrangement of the lock member 180, the button coupling rod 175, the actuation rod 109, and the spring 150 guarantees that there are only two stable positions for the lock member 180; a position in which axial motion of the actuation rod 109 is allowed while proximal motion of the dial 130 is prevented, and a position in which axial (and rotational) motion of the dial 130 is allowed while distal motion of the actuation rod 109 is prevented. In the former position the lock member 180 is retained by the actuation rod 109 until the actuation rod 109 during proximal movement relative to the housing 102 reaches the position where the transversal surface 198 passes the fulcrum of the lock member 180, at which point the lock member 180 pivots into locking abutment with the transversal surface 198 due to the distally directed force on the button coupling rod 175 provided by the spring 150 via the dial 130 and the double rack and pinion structure. In the latter position the lock member 180 is retained by the button coupling rod 175 until the button coupling rod 175 during proximal movement relative to the housing 102 reaches the position where the end surface 179 passes the fulcrum of the lock member 180, at which point the lock member 180 pivots into locking abutment with the end surface 179 due to the distally directed force on the actuation rod 109 provided by the spring 150.

1. A dosing unit for an injection device, the dosing unit comprising:
a housing extending along a longitudinal axis from a proximal housing end to a distal housing end, the distal housing end being adapted for connection with a variable volume reservoir,
an injection mechanism for expelling a dose of a substance from a connected variable volume reservoir, the injection mechanism being arranged at least partially in the housing and comprising:
an injection button,
a dose defining structure configured to move from a start position to an end-of-dose position in response to a dose expelling operation of the injection button, and
a piston rod configured to move in a dose delivery direction from a first position to a second position in response to a movement of the dose defining structure from the start position to the end-of-dose position, and
a dose delivered indicator configured to move axially in a distal direction from a dose ready position to a dose delivered position in response to the movement of the dose defining structure from the start position to the end-of-dose position,
wherein during movement from the dose ready position to the dose delivered position the dose delivered indicator emerges increasingly from the distal housing end.

2. A dosing unit according to claim 1, wherein the dose delivered indicator forms part of the injection mechanism and is operatively coupled with the piston rod during dose expelling such that rotational motion is imparted to the piston rod in response to the dose delivered indicator undergoing translational motion relative to the housing, and
wherein the piston rod is further threadedly engaged with a nut member fixedly arranged in the housing.

3. A dosing unit according to claim 1, further comprising a spring structure arranged to bias the dose delivered indicator in the distal direction.

4. A dosing unit according to claim 1, wherein at least one dose related indicium is arranged on the dose delivered indicator.

5. A dosing unit according to claim 4, wherein the at least one dose related indicium comprises a scale indicating progression in the proximal direction.

6. A dosing unit according to claim 1, wherein the dose delivered indicator is further movable from the dose delivered position to the dose ready position, during which movement the dose delivered indicator and the piston rod are operatively decoupled.

7. A dosing unit according to claim 1, further comprising a dose setting button operable to bring the dose defining structure to the start position,
wherein the dose defining structure comprises a plurality of dose setting indicia for indicating a size of a set dose, and
wherein the housing comprises a window enabling inspection of at least one of the plurality of dose setting indicia at a time.

8. A dosing unit according to claim 7, wherein at least a subset of the dose setting indicia successively pass by the window during movement of the dose defining structure from the start position to the end-of-dose position, thereby continuously indicating a remaining dose to be expelled.

9. An injection device comprising:
a dosing unit according to claim 1,
a variable volume reservoir comprising an outlet and a movable wall, and
a reservoir holder for connecting the variable volume reservoir with the distal housing end, the reservoir holder being configured to retain the variable volume reservoir in an axially fixed position relative to the housing in which the piston rod abuts the movable wall.

10. An injection device according to claim 9, further comprising at least one dose delivered indicium arranged on the variable volume reservoir or on the reservoir holder, distally of the distal housing end.

11. An injection device according to claim 10, wherein the at least one dose delivered indicium comprises a dose delivered scale indicating progression in the distal direction, and
wherein the dose delivered indicator is configured to slide along the dose delivered scale during movement from the dose ready position to the dose delivered position.

12. An injection device according to claim 9, wherein the reservoir holder comprises a longitudinal groove adapted to slidably receive the dose delivered indicator.

13. An injection device according to claim 9, further comprising a protective cap removably mountable onto the housing,
wherein the protective cap is structured to interface with the dose delivered indicator and move the dose delivered indicator from the dose delivered position to the dose ready position during mounting onto the housing.

14. An injection device according to claim 13, wherein the protective cap is adapted to be mounted onto the distal housing end, a mounting comprising at least a relative axial motion between the protective cap and the housing, and wherein the dose delivered indicator comprises a distal abutment surface and the protective cap comprises a rim configured for abutment with the distal abutment surface during the relative axial motion between the protective cap and the housing.
